(19) United States
(12) Patent Application Publication
(10) Pub. No.: US 2022/0409093 A1
(43) Pub. Date: Dec. 29, 2022

(54) DEVICE AND METHOD FOR DETECTING A SURFACE SHAPE ON THE HUMAN OR ANIMAL BODY

(71) Applicant: BELLWALD TEC GMBH - NWS, Muttenz (CH)

(72) Inventors: Thomas Ruepp, Muttenz (CH); Joris Pascal, Münchenstein (CH); Ralf Schumacher, Seltisberg (CH)

(21) Appl. No.: 17/781,345

(22) PCT Filed: Nov. 27, 2020
(86) PCT No.: PCT/EP2020/083588

§ 371 Date: May 31, 2022

(30) Foreign Application Priority Data
Dec. 3, 2019 (CH) 01532/19

(51) Int. Cl. A61B 5/107; G01B 7/287

(52) U.S. Cl. CPC A61B 5/1077; G01B 7/287; A61B 2562/0223

(57) ABSTRACT

The invention relates to a system and a method for detecting a surface shape of a human or animal body surface. A plurality of magnetic field sensors (2) arranged relative to one another in a sensor field (1) is placed on a plurality of surface regions of the body surface. A magnetic field generator (7) generates a magnetic field in the vicinity of the sensor field, so that a plurality of surface signals belonging to the surface regions are generated by using the magnetic field sensors (2). The surface signals serve as position signals with regard to the orientation and position of the magnetic field sensors in the magnetic field, so that the surface shape of the body surface is determined from the plurality of position signals of the magnetic field sensors (2) by using a signal processing unit (5).

The invention relates to a system and a method for detecting a surface shape on the human or animal body, in particular for digitally detecting a three-dimensional shape of limbs of a human or animal body.

There is a great and very different need to be able to detect a spatial shaping of surfaces of a human or even animal body. For example, patients with malformations or malpositions are measured using optical methods in order to be able to produce and adapt individual treatment solutions such as splints, corsets and the like. For the production of prostheses, it is also necessary to know a three-dimensional shape of an extremity stump in order to be able to ensure an optimum fit of the prosthesis on the stump.

In therapeutic areas, and here especially in orthopaedic technology, there is, for example, a need to be able to carry out a patient scan while a therapist or surgeon performs a therapeutic action on the body or while the body is resting on supporting devices. For example, a scan is taken during the straightening of a spasmic deformity of the lower extremities in order to develop a splint for holding a straightened position.

Today's systems use for example plaster casts with later optical digitization, or optical digitizing systems are used which scan body regions visible from the outside by detecting light reflected at the regions.

When using optical scanning methods for digitizing a body surface during a therapeutic action on the body, a hand of the surgeon or a support device can come into conflict with the optical scanning. The digital scanning and modelling devices available in orthopaedic technology ignore this deficiency, such as the device for measuring limbs described in DE 19822956 A1.

It is therefore customary to rework regions which were covered during scanning and were therefore not measured, for example manually, or to supplement them by using extrapolation or reconstruction methods. Only after the measurement is done can the recorded surface data be processed into a closed surface model by elaborate and uncertain reconstruction of missing data, which can then be made available for digital production of orthopaedic aids.

In addition, in orthopaedics, transilluminating imaging methods, such as, for example, computer tomography or magnetic resonance tomography, are used in particular to detect and image the shaping of internal body components. However, such methods can be harmful to health due to radiation exposure and high field strengths, nevertheless often providing poor data quality. In addition, they are cumbersome and expensive to use and require a high expenditure of energy.

Smaller devices are disclosed, for example, in WO 2005/013814, where an optical scanning device for detecting parameters of internal body parts, such as organs, muscles, fat or other tissue, during an examination of the body parts by an operator is shown. Anomalies of the body parts, such as tumours, can be detected, but not the external shaping of body surfaces.

In addition, different scanning and data acquisition methods have been developed for other health and medical fields, but these are not suitable for the three-dimensional acquisition of body surfaces. In particular, textiles with integrated sensors have been developed, which can, for example, be worn on the body in the form of clothing and are known under the name Smart Electro-Clothing Systems. Such systems can detect biosignals, including e.g. bioelectric, biomagnetic or biochemical signals. This involves integrating sensors of different types into the textiles, such as, for example, accelerometers, gyroscopes and magnetometers. The sensor system sends the detected signals to a processing unit for evaluating and displaying health-relevant information.

From WO 2014/204323, for example, an extensible textile fabric with integrated capacitive sensors is known, with which a stretching of the fabric and thus a movement of a body part which causes the stretching can be detected. In addition, socks or gloves are known which have large-area sensor fields with electrical sensors in order to measure a pressure application and pressure distribution on a foot sole or palm. As mentioned, it is not possible with such smart electro-clothing systems to detect the three-dimensional shape of a body surface.

It is therefore a task of the present invention to provide a system and a method for detecting a surface shape on the human or animal body, which can reliably and completely detect this surface shape, which manage as far as possible without post-processing the detected surface data, which can be operated in a cost- and energy-saving manner and do not pose a risk to the body.

The invention is especially intended to provide a system and a method for detecting a three-dimensional shape of a body surface which allow a reliable statement to be made about a therapeutic approach to an orthopaedic aid, such as, for example, an orthotic shape.

According to the invention, this task is solved by a system according to Claim 1 and a method according to Claim 12. Advantageous embodiments and different variants of the invention emerge from the subclaims.

A system for detecting a surface shape of a human or animal body surface according to the present invention comprises a sensor field with a plurality of sensors which is designed to scan the body surface at a plurality of surface regions of the body surface and to generate a plurality of associated surface signals. In addition, the system has a signal processing unit which is designed to determine the surface shape of the body surface from the multiplicity of surface signals of the multiplicity of sensors. In this case, the sensors of the sensor field are each assigned to at least one surface region and supply a surface signal assigned to this region. The surface shape of the body surface can thus be determined from the totality of surface signals.

According to the invention, the system comprises a magnetic field generator for generating at least one magnetic field in the vicinity of the sensor field. Thus, a magnetic field is present in the vicinity of the body surface whose shape is to be determined. In addition, the sensor field is formed by a multiplicity of magnetic field sensors which are arranged relative to one another on the body surface. Thereby, the multiplicity of magnetic field sensors is assigned to the multiplicity of surface regions, for the magnetic field sensors to be able to be distributed along the body surface. The magnetic field sensors are designed to generate a position signal as a surface signal with respect to their orientation and position in the magnetic field. The position signal of a magnetic field sensor provides at least information relating to the spatial orientation of the sensor in the magnetic field. Preferably, the position signal also comprises information about the location of a sensor in the magnetic field. In addition, the position signal can provide information about the position of the sensor in the sensor field or relative to the other sensors. A position and orientation of a surface region of the body surface is thus represented by an associated magnetic field sensor. In this case, a surface region corresponds to at least one region of the body surface on which the magnetic field sensor rests. However, the surface region can also extend beyond this to the point where an adjacent surface region begins. The position signals of the magnetic field sensors thus provide a network of surface signals which can image the entire body surface and, if appropriate, an entire body peripheral surface. Thus, the signal processing unit may determine the surface shape of the body surface from the plurality of position signals and provide a closed surface model.

Advantageously, the signal processing unit digitizes the surface shape of the body surface, so that a digital data set of the detected body surface is present, which can be used directly for the treatment of a patient or for the development of medical aids, such as an orthosis. The system can thus detect any three-dimensional surface shape of a three-dimensional human or animal body surface. Advantageously, a closed body peripheral surface is detected, such as is present, for example, in the case of limbs.

The scanning of the body surface by the sensor field is effected by the coupling and excitation of the magnetic field sensors by the magnetic field of the magnetic field generator. Wide-area scanning is made possible by the expansion of the sensor field over the body surface.

The method for detecting a surface shape of a human or animal body surface according to the present invention preferably uses a detection system as described above. In the method according to the invention, the multiplicity of magnetic field sensors arranged relative to one another in a sensor field is placed on the multiplicity of surface regions of the body surface. In addition, a magnetic field is generated by the magnetic field generator in the vicinity of the sensor field. As a result, a multiplicity of surface signals belonging to the surface regions are generated by using the magnetic field sensors, the surface signals being in form of position signals with respect to the alignment and position of the magnetic field sensors in the magnetic field. The surface shape of the body surface is then determined by the signal processing unit from the multiplicity of position signals of the magnetic field sensors.

The system and the method according to the present invention can reliably and completely detect the surface shape, since treatments and support devices can easily act on the body surface above the sensor field without hindering the scanning of the position signals. It is thus also possible to scan body regions which are covered by a hand or an aid during the scanning. Subsequent processing of recorded surface data, for example for bridging over regions which have not been scanned, is not necessary, as a result of which precise and detailed recording of the body surface is possible.

In an advantageous embodiment, the plurality of magnetic field sensors is arranged in the sensor field in a regular grid. The sensors are preferably arranged in the grid at a predetermined grid spacing from one another. The grid spacing can be selected, for example, in accordance with a required accuracy of the surface detection. The sensor field can also be divided into sectors with different grid spacings. Thus, for example, in regions of the body surface with a greatly varying surface shape, smaller distances can be selected than in regions with a more uniform surface shape. Typically, a distance between the magnetic field sensors is in the range of 1 mm to 500 mm, preferably in the range of 5 mm to 50 mm. With large distances of, for example, 50 mm to 500 mm, large body surfaces can be scanned e.g. of large animals. Material for the sensors and their interconnection is also saved when large distances are used.

A grid spacing of grid rows in the sensor field is preferably variable. This means that the grid spacing can be increased or decreased, depending on the conditions of a body surface or a movement of the body. The grid spacing of the sensor field can also change when the magnetic field sensors are placed on the surface of the body, in order to make it possible to accurately mount the magnetic field sensors on the surface.

Advantageously, the plurality of magnetic field sensors of the sensor field are arranged relative to one another in a deformable sensor plane. The entirety of the magnetic field sensors arranged next to one another thus forms a plane of sensors. This plane can be deformed by moving adjacent sensors relative to one another. The sensor plane is thus capable of conforming to a body surface, so that the shape of the plane corresponds to the shape of the body surface. In order to span this plane, two or more sensors can be connected to one another, for example by flexible connecting means, such as, for example, threads or cables. It is also possible to connect the sensors to one another by using electrical lines for tapping the surface signals. The sensor plane can also be self-contained and form a kind of circumferential plane.

In one embodiment, the plurality of magnetic field sensors of the sensor field or a part of the plurality of magnetic field sensors are connected to one another in series. This means that the magnetic field sensors are connected to one another linearly, or in a row. One magnetic field sensor is connected to a maximum of two other sensors. The magnetic field sensors connected to one another in series form a type of sensor chain which can be laid out and form the sensor plane. Thereby, the sensor chain can run, for example, in a straight line, in curves or in loops. Alternatively, the magnetic field sensors can form a type of sensor network where at least some of the magnetic field sensors are connected to at least three other sensors. The sensor network can then span the sensor plane.

In a preferred embodiment, the plurality of magnetic field sensors of the sensor field is mounted in or on a textile material. To be able to detect the shape of the body surface, the textile material with the magnetic field sensors is placed on a body surface in such a way that the multiplicity of magnetic field sensors come to lie on the associated multiplicity of surface regions of the body surface. In this case, the magnetic field sensors align their position in accordance with an orientation of an associated surface region. The textile material can thus serve for the simple mounting of the magnetic field sensors relative to one another and also for a simple and pleasant placement of the sensors on the body surface. Due to the flexibility of the textile material, the material adapts to the surface of the body and automatically positions the magnetic field sensors on the surface in accordance with the orientation of the surface.

The textile material is preferably designed to be stretchable. The sensor field can thus be stretched over a body surface, in particular around a body peripheral surface, and is held securely in position there. In addition, the textile material can be stretched over the surface of the body in such a way that a predetermined distance between the magnetic field sensors changes. The shape of the body surface can be inferred from the change in the predetermined distance. The change can thus be a component of the position signal of the magnetic field sensors.

In a simple variant of the system according to the present invention, the textile material is designed, for example, as a textile cloth with the magnetic field sensors, which can be placed on or around a body surface. However, the textile material with the magnetic field sensors can also be processed, for example, in a sock, a stocking, a glove, sleeves or the like, so that the patient can put the textile material on via his limbs. Alternatively, the textile material can also be used in a glove which is worn by an operator. The operator can use the glove to scan a body surface of the patient to be measured. The operator places his or her hand with the glove on the surface of the body. The operator can also feel the surface of the body with the glove.

The magnetic field sensors are designed as three-dimensional magnetic field sensors. In one embodiment of the system for detecting a surface shape of a human or animal body surface according to the present invention, the magnetic field sensors are designed as Hall sensors. With Hall sensors, the magnetic flux density of the magnetic field generated by the magnetic field generator can be measured and the position of a sensor in the magnetic field and relative to other sensors can be determined therefrom. In addition, magnetoresistive (MR) sensors or sensors in the form of an electronic compass, for example TMR, AMR or GMR sensors, can be used as magnetic field sensors. A mixture of different types of sensors is also possible.

In one embodiment, the magnetic field sensors each comprise two or more sensor units which are designed and arranged for a three-dimensional measurement. For example, two or more sensor units are provided, which are arranged perpendicular to one another. The magnetic field sensors detect the field vector information which belong to their orientation and position in the magnetic field.

In one embodiment of the system according to the present invention, the magnetic field generator is designed as a three-axis magnetic field source. For example, electromagnets, a coil or movable permanent magnets can be used as the magnetic field source. The three-axis magnetic field source can be used to generate three superimposed magnetic fields. Each of the magnetic fields can be detected by the magnetic field sensors, so that a resulting position signal of the sensors comprises three spatial components, as a result of which precise position information is obtainable for each sensor. As an alternative to a three-axis magnetic field source, three individual magnetic field sources can, for example, also be arranged in a triangle to each other. The three sources form a superimposed total magnetic field for measurement with the magnetic field sensors.

Advantageously, the magnetic field generator generates a magnetic field having a strength of 10 nT, preferably 100 nT to 100 mT, in the vicinity of the sensor field. In a variant of the detection system for scanning a forearm, for example, 2 mT were used.

Compared to conventional magnetic imaging methods for detecting surface shapes, such as, for example, magnetic resonance tomography, the magnetic field used in the method according to the present invention is smaller by a factor of 10 to 30 million. The process according to the invention therefore requires less energy and is more cost-effective.

In a further embodiment of the system for detecting a surface shape of a human or animal body surface according to the present invention, the sensor field has a further multiplicity of sensors in the form of pressure and/or temperature sensors. These sensors are designed to determine a signal corresponding to a pressure and/or a temperature at a multiplicity of surface regions of the body surface. In this case, the surface regions of these sensors do not have to coincide with the regions which are assigned to the magnetic field sensors. In addition, it is not necessary to provide the same number of regions for these sensors as for the magnetic field sensors.

However, the magnetic field sensors and the pressure and/or temperature sensors are advantageously combined to form a sensor component and can be provided jointly in the sensor field and at surface regions assigned to the magnetic field sensors. The pressure and/or temperature sensors can be used to determine pressure and/or temperature signals at the associated surface regions. In combination with the position signals of the magnetic field sensors, an areal pressure and/or temperature distribution on the body surface can thus be determined.

With the system and the method for detecting a surface shape of a human or animal body surface according to the present invention, a digital data set of a peripheral surface of the human or animal body surface can thus be produced from the multiplicity of position signals. As an example, the fineness of the data grid is in the range of 1 mm to 50 cm for large animals and in the range of 1 mm to 10 cm for humans. The system is constructed from simple components and has a portable size. This allows the method to also be used on an outpatient basis. The detection method is simple to use, poses no health risks due to low magnetic field strengths and can be operated cost-effectively. Especially for the construction and adaptation of orthopaedic aids such as splints, orthoses, prostheses and the like, it is advantageous that all regions of a body peripheral surface can be scanned and thus no post-processing of the acquired data sets is required. In this way, the fit and wearing comfort of aids can be significantly improved.

The invention is illustrated below with the figures, which serve merely to explain and are not to be interpreted in a restrictive manner. The features of the invention which become apparent from the figures need to be regarded individually and in any combination as belonging to the disclosure of the invention. What is shown is:

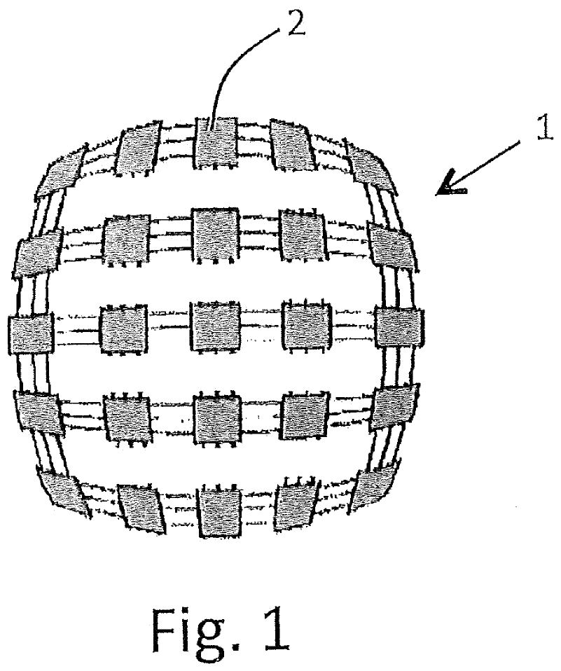

FIG. 1 is a schematic representation of a first variant of a sensor field of a system for detecting a surface shape of a human or animal body surface according to the present invention,

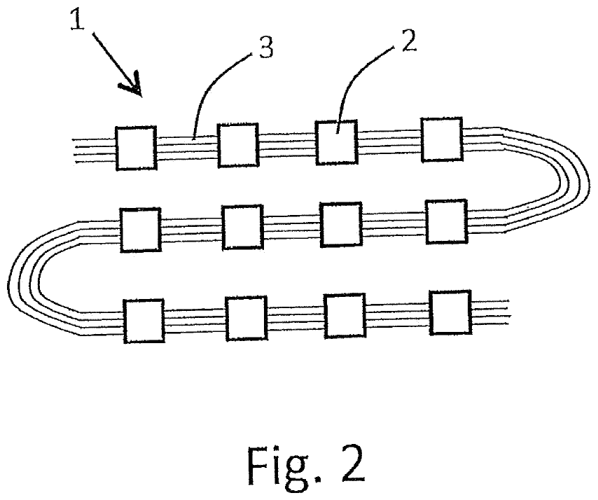

FIG. 2 is a schematic representation of a second variant of a sensor field of a system for detecting a surface shape of a human or animal body surface according to the present invention, and

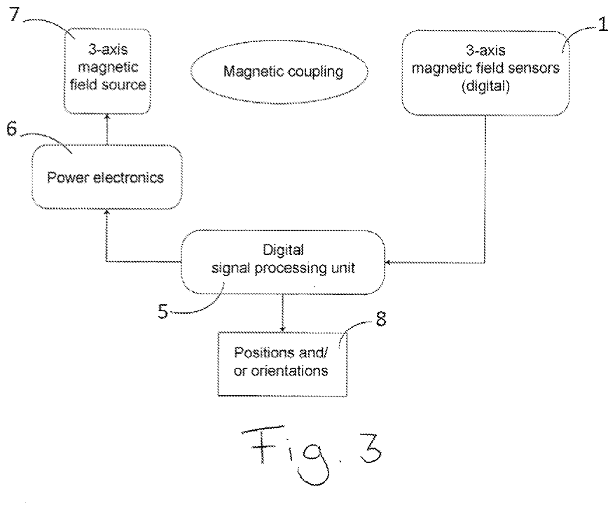

FIG. 3 is a flow chart of a method for detecting a surface shape of a human or animal body surface according to the present invention.

As shown in FIG. 1, according to the invention a system for detecting a surface shape of a human or animal body surface is provided which comprises a sensor field with a plurality of magnetic field sensors. The sensor field is provided for covering a three-dimensional body surface, so that the body surface is subdivided into a multiplicity of surface regions. Each of the magnetic field sensors scans the surface region assigned to it and generates a position signal with information on its position on the body surface. Thus, a plurality of surface signals corresponding to the plurality of magnetic field sensors and associated surface regions are generated.

As can be seen in FIG. 1, the sensor units are integrated into a carrier 4 in the form of a flexible or extensible printed circuit board (PCB). The magnetic field sensors 2 are thus provided as three-axis magnetic field sensors. The magnetic field sensors 2 each generate a position signal with information about the orientation of the respective sensor with respect to the body surface and advantageously also about the local position of the magnetic field sensors to each other.

As can be seen from FIG. 1, the magnetic field sensors 2 are arranged in the sensor field 1 in a grid with predetermined regular distances from one another. The grid can be realized, for example, by attaching the magnetic field sensors in a textile material (not shown). Conductors (not shown) for relaying digital position signals of the magnetic field sensors may also be provided in or on the textile material. The sensors are advantageously connected in series, so that not all sensors are connected to one another and material can be saved.

In an advantageous variant of the detection system according to the invention, the sensor field forms a deformable sensor plane, as is indicated in FIG. 1. For this purpose, for example, the textile material on which the magnetic field sensors are arranged can be flexible and stretchable, so that the orientation of the individual magnetic field sensors to each other and also their distance from one another are variable. The sensor plane can thus adapt to any desired shape of a body surface and align the magnetic field sensors in accordance with the orientation of the respective surface regions. As indicated in FIG. 1c, the sensor plane shown is, for example, bulged outwardly from the plane of the drawing.

FIG. 2 shows an alternative embodiment of a system for detecting a surface shape of a human or animal body surface, where the magnetic field sensors are placed in series. The magnetic field sensors 2 are connected in series with four lines 3 and form a kind of sensor chain. The sensor chain can in turn be attached to a textile material. In the example shown, the sensor chain is bent in such a way that three essentially parallel rows result, which together form a sensor plane. In this way, a large number of magnetic field sensors and only a few lines are provided in the system.

FIG. 3 shows a diagram for carrying out a method for detecting a surface shape of a human or animal body surface according to the present invention. A signal processing unit 5 sends an activation signal to power electronics 6 in order to activate a magnetic field generator 7. In this variant, the magnetic field generator 7 is provided, for example, as a three-axis magnetic field source, such as, for example, in the form of a three-axis coil.

The magnetic field generator 7 generates a magnetic field in the vicinity of the sensor field 1. The magnetic field thus surrounds the multiplicity of magnetic field sensors 2, which belong to the multiplicity of surface regions of the body surface, and triggers a magnetic excitation of the magnetic field sensors 2. The magnetic field sensors 2 generate a surface signal in response to the magnetic field excitation in accordance with the magnetic field strength and their angular orientation and position in the magnetic field, which in turn depend on their orientation in accordance with the body surface and their distance from one another. This information can be determined on the basis of physical laws relating to the decrease in the magnetic field strength with the distance to the magnetic field source and the angular orientation in the magnetic field. This information is then sent by the plurality of magnetic field sensors 2 as a plurality of position signals to the signal processing unit 5. The position signals thus map the orientation and the extent of the surface regions, so that the signal processing unit 5 can determine the shape of the body surface therefrom and output it as a digital data set 8 of the surface shape. This digital data set can, for example, be fed directly to a CAD program for constructing an orthopaedic aid.

Although the detection method has been explained with reference to an example of a static surface shape determination, it is, with the system and the method according to the present invention, quite possible to carry out a dynamic shape determination where the shape of a body surface changes, for example, in accordance with a movement of a patient during the scanning. It is merely necessary to ensure that the patient moves within the magnetic field generated by the magnetic field generator. As a result, it is possible, for example, to determine the snug fit of orthopaedic aids during a movement of the patient.

The system and the method for detecting a surface shape of a human or animal body surface according to the invention thus have wide-ranging possible applications and can at the same time be used in a cost-effective and energy-saving manner. In addition, they permit a high degree of accuracy of the surface shapes detected. In particular, a closed surface model can be produced, for example, around a hand, an arm, a foot and the like, as is frequently required in orthopaedic technology.

### REFERENCE SYMBOL LEGEND

1 Sensor field
2 Magnetic field sensors
3 Lines
4 Beams
5 Signal processing unit
6 Power electronics
7 Magnetic field generator
8 Digital dataset

1. A System for detecting a surface shape of a human or animal body surface, which
comprises a sensor array (1) with a plurality of sensors, which is designed to scan the body surface at a plurality of surface areas and to generate a plurality of associated surface signals, and
a signal processing unit (5) adapted to determine the surface shape of the body surface from the plurality of surface signals of the plurality of sensors, characterized by
a magnetic field generator (7) for generating at least one magnetic field in the vicinity of the sensor field (1), and
a plurality of magnetic field sensors (2) which form the sensor field (1) and are arranged with respect to one another so as to rest on the surface of the body, the magnetic field sensors (2) being designed to generate, as a surface signal, a position signal with regard to their orientation and position in the magnetic field,
so that the signal processing unit (5) determines the surface shape of the body surface from the plurality of position signals.

2. The System according to claim 1, wherein the plurality of magnetic field sensors (2) are arranged in the sensor field (1) in a regular grid.

3. The System according to claim 1, wherein the multiplicity of magnetic field sensors (2) of the sensor field (1) are arranged in relation to one another in a deformable sensor plane.

4. The System according to claim 1, wherein cat least a part of the plurality of magnetic field sensors (2) of the sensor field (1) are connected in series with each other.

5. The System according to claim 1, wherein the plurality of magnetic field sensors (2) of the sensor field (1) are mounted in or on a textile material.

6. The System according to claim 5, wherein the textile material is stretchable.

7. The System according to claim 1, wherein the magnetic field sensors (2) are designed as Hall or as magnetoresistive sensors.

8. The System according to claim 1, wherein the magnetic field sensors (2) comprise two or more sensor units designed and arranged for a three-dimensional measurement.

9. The System according to claim 1, wherein the magnetic field generator (7) is designed as a three-axis magnetic field source.

10. The System according to claim 1, wherein the magnetic field generator (7) generates a magnetic field of strength 10 nT to 100 mT in the vicinity of the sensor field (1).

11. The System according to claim 1, wherein the sensor array (1) comprises a further plurality of pressure and/or temperature sensors adapted to determine a signal corresponding to a pressure and/or a temperature at a plurality of surface areas.

12. A method of detecting a surface shape of a human or animal body surface comprising:
placing a plurality of magnetic field sensors (2) arranged relative to one another in a sensor field (1) on a plurality of surface regions of the body surface,
generating at least one magnetic field in the vicinity of the sensor field (1),
generating a plurality of surface signals belonging to the surface areas by using the magnetic field sensors (2), the surface signals being in the form of position signals with respect to the orientation and position of the magnetic field sensors in the magnetic field, and
determining the surface shape of the body surface from the plurality of position signals of the magnetic field sensors (2) by using a signal processing unit (5).

13. The Method according to claim 12, wherein the magnetic field sensors (2) are mounted in or on a textile material which is placed on a body surface in such a way that the plurality of magnetic field sensors (2) come to lie on a plurality of surface regions of the body surface, the magnetic field sensors (2) aligning their position according to an orientation and position of an associated surface region.

14. The method of claim 13, wherein the textile material is stretched over the body surface so that a predetermined distance of the magnetic field sensors (2) from each other changes.

15. The Method according to claim 12, wherein a digital data set (8) of a peripheral surface of the human or animal body surface is created from the plurality of position signals.

16. The system of claim 2, wherein a grid spacing of grid rows is variable.
